(19) United States
(12) Patent Application Publication
(10) Pub. No.: US 2016/0302829 A1
(43) Pub. Date: Oct. 20, 2016

(54) GUIDE DEVICE FOR INSERTION OF BLOCKING SCREWS USED TO ALIGN INSERTION OF INTRAMEDULLARY NAIL

(71) Applicant: Rahul Vaidya, Ann Arbor, MI (US)

(72) Inventor: Rahul Vaidya, Ann Arbor, MI (US)

(21) Appl. No.: 14/690,467

(22) Filed: Apr. 19, 2015

(51) Int. Cl. A61B 17/56; A61B 17/72

(52) U.S. Cl. CPC A61B 17/56; A61B 17/725; A61B 2017/564; A61B 2017/90

(57) ABSTRACT

A blocking screw insertion guide useful for proper alignment of blocking screws used in intramedullary nail fixation of fractured long bones. The insertion guide including a intramedullary nail dummy head consisting of a short segment of the proximal portion of an intramedullary nail; a nail insertion handle of the type used to insert intramedullary nails; and a blocking screw guide portion. The invention also includes a method of intramedullary nail fixation of fractured long bones using the inventive blocking screw insertion guide.

### FIELD OF THE INVENTION

The present invention relates to the use of intramedullary nails for the repair of long bone fractures. More specifically, the present invention relates to the insertion of blocking screws used to align the insertion of the intramedullary nail. Most specifically, the present invention comprises a guide device useful than to provide proper insertion of the blocking screws and a method of using the inventive guide device in the repair and setting of long bone fractures.

### BACKGROUND OF THE INVENTION

Rod or nail placement in the medullary cavity for securing bone fractures is a common practice in orthopedic surgery. Use of the rod is known to inherently produce better healing in more extreme fractures than other procedures in which the rod is omitted. Spanning the fracture zone, the rod imposes a rigidity to the fracture area that could otherwise be difficult to maintain during the prolonged period of mending. When left permanently in place, the rod reinforces the bone and reduces its susceptibility to refracture. Use of the rod is known to inherently produce better healing in more extreme fractures than other procedures in which the rod is omitted.

An intramedullary rod, also known as an intramedullary nail (IM nail) or inter-locking nail or Küntscher nail, is a metal rod forced into the medullary cavity of a bone. IM nails have long been used to treat fractures of long bones of the body. Gerhard Küntscher is credited with the first use of this device in 1939, during World War II, for soldiers with fractures of the femur. Prior to that, treatment of such fractures was limited to traction or plaster, both of which required long periods of inactivity. IM nails resulted in earlier return to activity for the soldiers, sometimes even within a span of a few weeks, since they share the load with the bone, rather than entirely supporting the bone.

The earliest IM nails were triangular or ‘V’ shaped in cross-section. Later they were modified to their present and more rotationally stable clover-leaf shape. Several modifications and shapes were introduced subsequently for various bones such as V-nails for tibia, radius and ulna nails, Rush nails etc.

Steel continues to be the material of choice for these nails, owing to its strength, but more recently titanium has gained popularity due to its advantages over steel. However the biggest problem with the earlier designs was the failure to prevent collapse or rotation in inherently unstable fractures. This was addressed by the introduction of the concept of ‘locking’ of the nails using screws on each end of the nail, leading to emergence of locked IM nailing, which is the standard today.

During insertion of the intramedullary nail, these sections of the fractured bone may become misaligned which, if left unmediated, could result in improper healing of the fractured bone. The incidence of malalignment is reported to be as high as 58% for proximal tibia fractures, 14% for distal tibias, 30% for proximal femurs and 10% for distal femoral fractures. Blocking screws (also known as Poller screws), aid in obtaining satisfactory alignment during surgery and provide additional stability. When strategically placed, these screws guide the reamers and the nail to a suitable trajectory, thereby achieving indirect reduction. They also reduce the size of the medullary cavity and increase the stiffness of the bone-implant construct. Unfortunately, proper insertion of the blocking screws can be difficult, and may require more than one attempt to properly align the screws for implantation of the intramedullary nail. There exists in the art a need for a device to easily align and insert the blocking screws into the long bone to provide proper guide for insertion of the intramedullary nail.

### SUMMARY OF THE INVENTION

A first invention is a blocking screw insertion guide useful for proper alignment of blocking screws used in intramedullary nail fixation of fractured long bones. The insertion guide may include an intramedullary nail dummy head which may consist of a short segment of the proximal portion of an intramedullary nail. The insertion guide may further include a nail insertion handle of the type used to insert intramedullary nails. The insertion guide may also include a blocking screw guide portion. The dummy head may be connected to the nail insertion handle in the same manner that an actual intramedullary nail would be connected for insertion into the fractured long bone. The blocking screw guide portion may be attached to the nail insertion handle and may include means to align/guide insertion of blocking screws into proper positions to substantially reduce or eliminate the incidence of malalignment of the fractured bone during intramedullary nail fixation thereof.

The blocking screw guide portion may include a guide attachment means and may also include a blocking screw guide. The guide attachment means may connect the nail insertion handle and the blocking screw guide. The blocking screw guide may include means to align/guide insertion of blocking screws into proper positions to substantially reduce or eliminate the incidence of malalignment of the fractured bone during intramedullary nail fixation thereof. The means to align/guide insertion of blocking screws may comprise alignment holes in the blocking screw guide. The blocking screw guide may be a curved or arced plate. The dummy head may be connected to the nail insertion handle by a screw or bolt. The guide attachment means may be attached to the nail insertion handle by one or more screws or bolts.

A second invention is an intramedullary nailing method for the fixation of fractured long bones which substantially reduces or eliminates the incidence of malalignment of the of fractured long bones. The method may include the step of selecting the proper intramedullary nail for the specific long bone fracture. The method may further include the step of providing the blocking screw insertion guide of the first invention described herein above. The inventive method may further include the step of inserting the dummy head of the blocking screw insertion guide into the medullary cavity of the fractured long bone and may also include the step of using the blocking screw guide portion to insert blocking screws into the fractured long bone.

The intramedullary nailing method may further comprise the step of removing the dummy head of the blocking screw insertion guide from the medullary cavity of the fractured long bone after the step of inserting the blocking screws. The method may also include the step of inserting the selected proper intramedullary nail into the fractured long bone, the insertion of the selected proper intramedullary nail being guided by the blocking screws such that the incidence of malalignment of the fractured long bone is substantially reduced or eliminated after the step of removing the dummy head of the blocking screw insertion guide from the medullary cavity of the fractured long bone. The inventive intramedullary nailing method may further comprise the step of locking the selected proper intramedullary nail into the medullary cavity of the fractured long bone using conventional locking screws or pins after the step of inserting the selected proper intramedullary nail into the fractured long bone. Finally, the method may include the further step of removing the blocking screws after the step of locking the selected proper intramedullary nail into the medullary cavity of the fractured long bone.

### BRIEF DESCRIPTION OF THE DRAWINGS

FIG. 1 depicts an extension malalignment of a long bone fracture which was caused by conventional intramedullary nail fixation without the use of blocking screws;

FIG. 2 depicts one embodiment of the blocking screw insertion guide of the present invention;

FIG. 3 depicts a side view of the blocking screw insertion guide means of the device of FIG. 2.

FIG. 4 illustrates the method in which the blocking screw insertion device is used to insert blocking screws into the fractured long bone prior to intramedullary nail fixation, specifically shown is the manner in which the dummy nail head is inserted into the medullary cavity;

FIG. 5 shows a cross-section of the fractured long bone with the dummy nail head inserted into the medullary cavity, note that the guide attachment means and the blocking screw guide are now in position to allow for proper insertion of blocking screws through the alignment holes;

FIG. 6 depicts inserted blocking screws which have passed through the alignment holes in the blocking screw guide and into the fractured long bone;

FIG. 7 shows a fractured long bone which has been fixated by an intramedullary nail guided by blocking screws which were inserted using the blocking screw insertion device of the present invention; and

FIG. 8 shows an intramedullary nail fixated femur fracture in which the nail has been guided by blocking screws inserted using the blocking screw insertion device of the present invention.

### DETAILED DESCRIPTION OF THE INVENTION

The present invention is a guide device that will work with any nailing system. In its most simplistic form, it is a short segment of the proximal portion of any intramedullary nail that is attached to a nail insertion handle and a custom guide for that nail. The dummy portion of the nail is a very important piece to the inventive device and method. This is because when inserted, long nails are often mal aligned as they pass from one fragment of the bone to the next. Using the dummy portion in the proximal or insertional piece of the long bone allows the surgeon to place it in the optimal position, then place blocking screws around it to make sure the real nail sits in this position as well. This would not be possible without the dummy nail. The dummy nail can take the shape of any commercially available nail but the guide would be modified to fit each different nail. This device can be made for the tibia, the distal femur, the proximal femur and the proximal humerus. The present invention allows blocking screws to easily be placed in key positions.

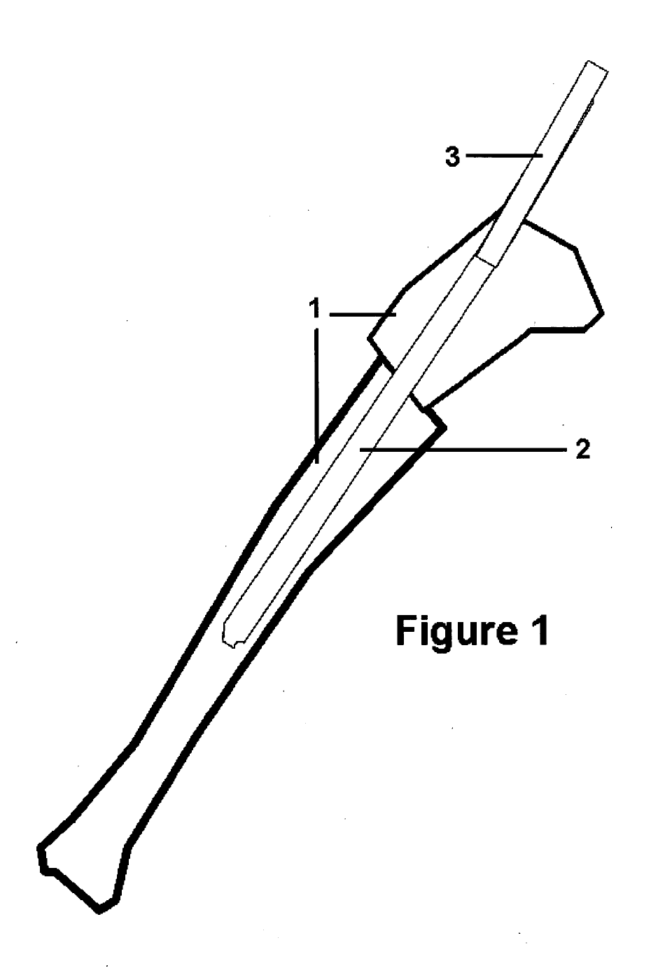

As a first example of an embodiment of the present invention the repair of a proximal tibia fracture is considered. The proximal tibia fracture is often difficult to nail, because the proximal portion ends up in extension if it is nailed in the traditional manner. FIG. 1 depicts an example of the extension malalignment of a fractured long bone 1 which has been fixated in the traditional manner with an intramedullary nail 2 attached to a nail insertion handle 3. Short plates, Schanz pins or clamps have been used in the past to solve this problem. Only the plate works well.

Blocking screws also are a useful method of controlling the extension misalignment. However, correct placement of blocking screws to ensure properly aligned fixation of the long bone fracture is difficult, time-consuming and may require more than one attempt to properly align the screws for implantation of the intramedullary nail.

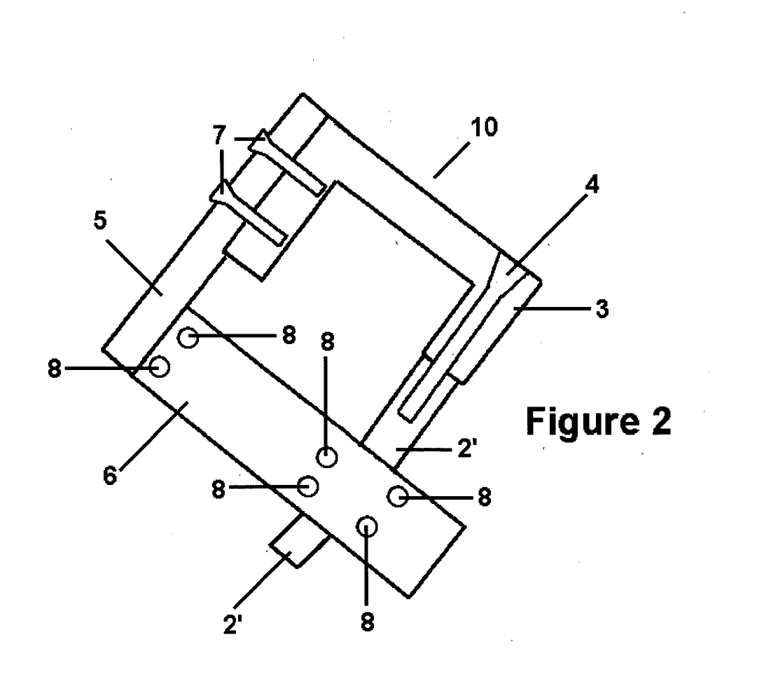

FIG. 2 depicts one embodiment of the present invention, namely, a device to easily align and insert the blocking screws into the long bone to provide proper guide for insertion of the intramedullary nail. The blocking screw insertion device 10 consists of three basic components. The first component is a short segment of the proximal portion of any intramedullary nail hereinafter referred to as the dummy nail head 2′. The dummy nail head 2′ is attached to the second component, a nail insertion handle 3. The nail insertion handle 3 is the specific handle which is designed and sold for use with the actual intramedullary nail to be used in the fixation of the long bone fracture. The nail insertion handle 3 is affixed to the dummy nail head 2 via any attachment means 4 such as a screw or bolt. Attached to the nail insertion handle 3 is the blocking screw insertion guide means. In this specific example, the blocking screw insertion guide means comprises two components. First, and attachment means 5, which is attached to the nail insertion handle 3 via any affixing means such as screws 7. Blocking screw guide 6 is attached to attachment means 5. Blocking screw guide 6 includes guide means such as alignment holes 8. The alignment holes 8 are designed such that when blocking screws are passed there through and into the fractured bone, the blocking screws are properly placed to ensure proper alignment of the subsequently nailed fracture.

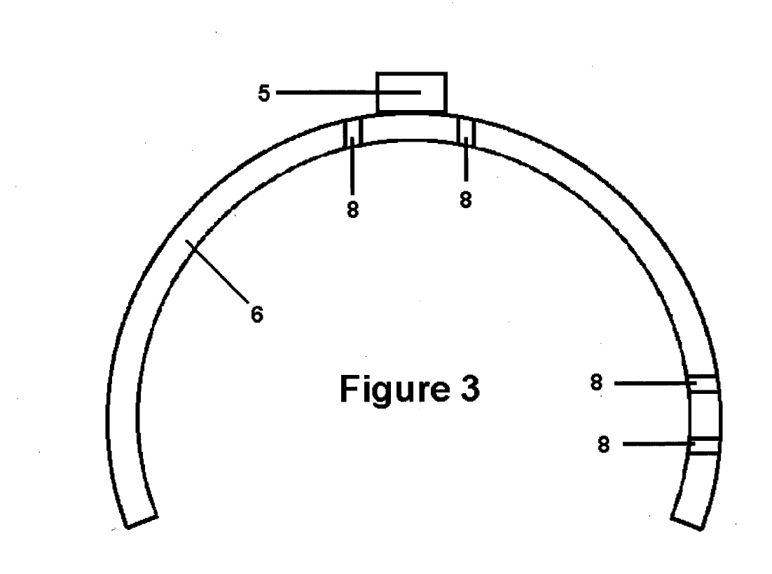

FIG. 3 is a side view of the blocking screw insertion guide means of the device of FIG. 2. As can be seen, in this embodiment, the guide attachment means 5 consists of a simple bar. However any other attachment means may be used, as long as it allows for adequate and accurate attachment of blocking screw guide 6. In this embodiment, the blocking screw guide 6 is in the form of an arced plate, which has sets of alignment holes 8 positioned at approximately right angles to one another. In practice, the blocking screw alignment guide 6 may have other shapes and configurations in the sets of alignment holes 8 may be at other than right angles.

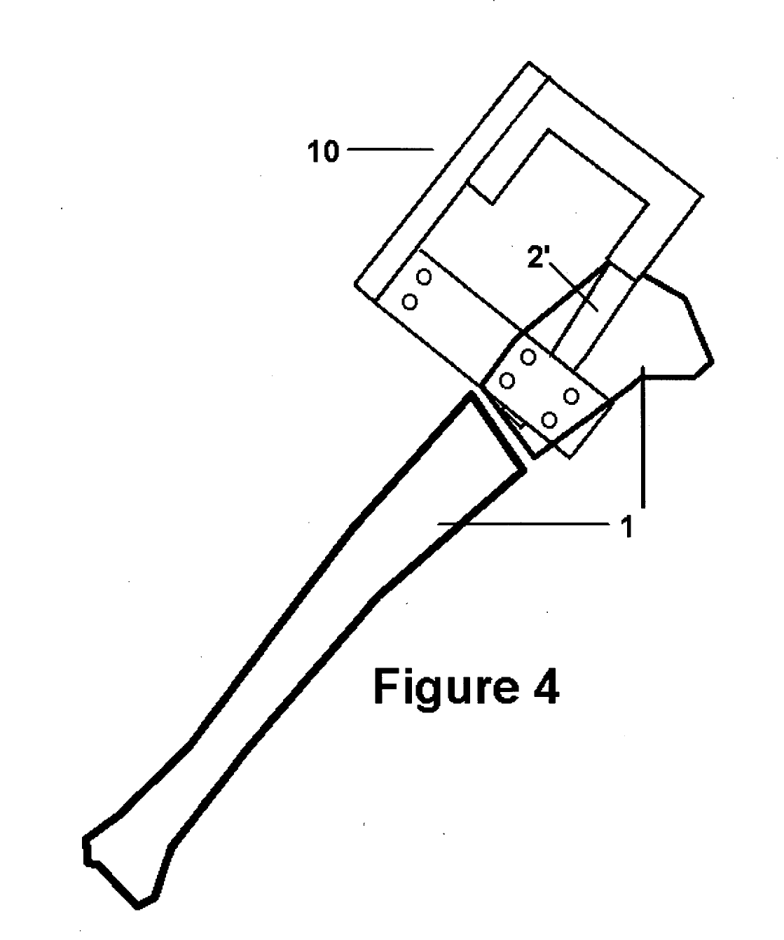

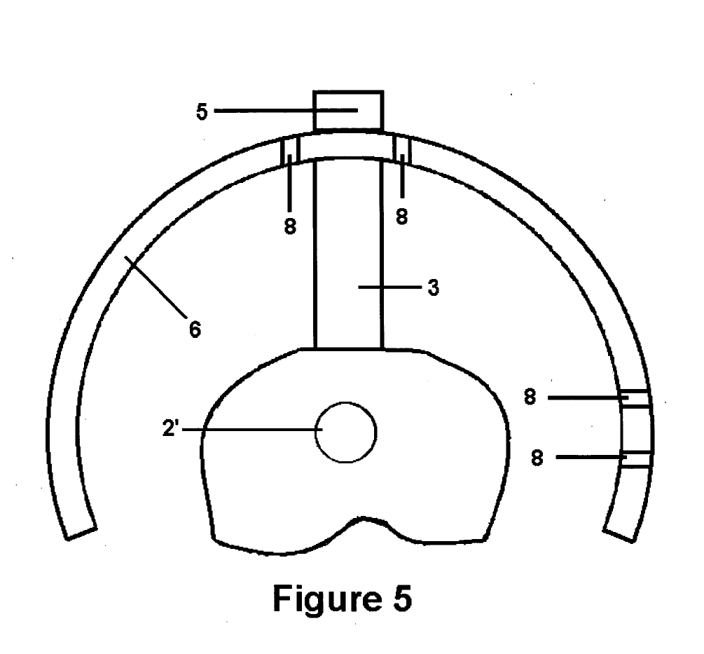

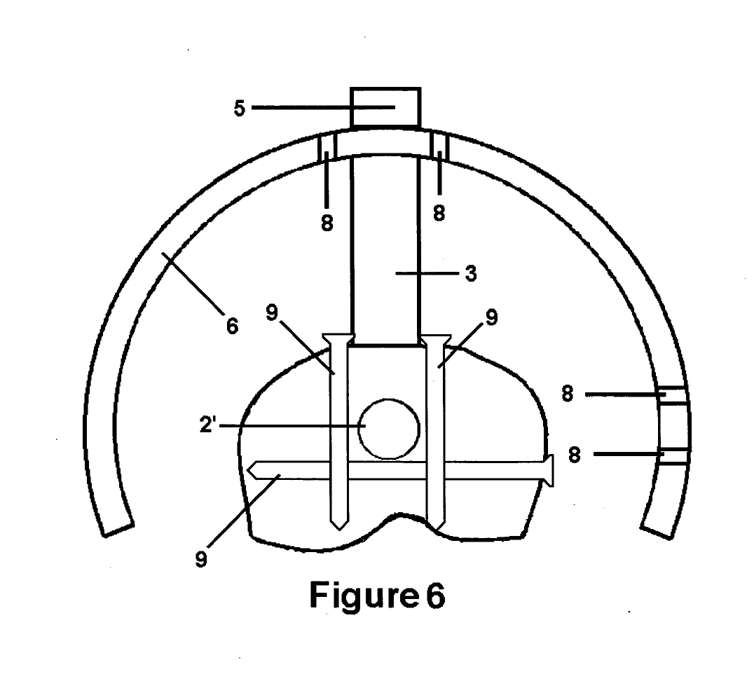

FIG. 4 illustrates the method in which the blocking screw insertion device 10 is used to insert blocking screws into the fractured long bone 1 prior to intramedullary nail fixation. The dummy nail head 2′ is inserted into the medullary cavity. FIG. 5 shows a cross-section of the fractured long bone 1 with the dummy nail head 2′ inserted into the medullary cavity using the nail insertion handle 3. The guide attachment means 5 and the blocking screw guide 6 are now in position to allow for proper insertion of blocking screws through alignment holes 8. FIG. 6 depicts inserted blocking screws 9, which have passed through alignment holes 8 in the blocking screw guide 6. The medial to lateral screw (horizontal in FIG. 6) controls the nail from heading too far back and prevents the extension misalignment deformity, while the AP screws (vertical in FIG. 6) control varus and valgus misalignment.

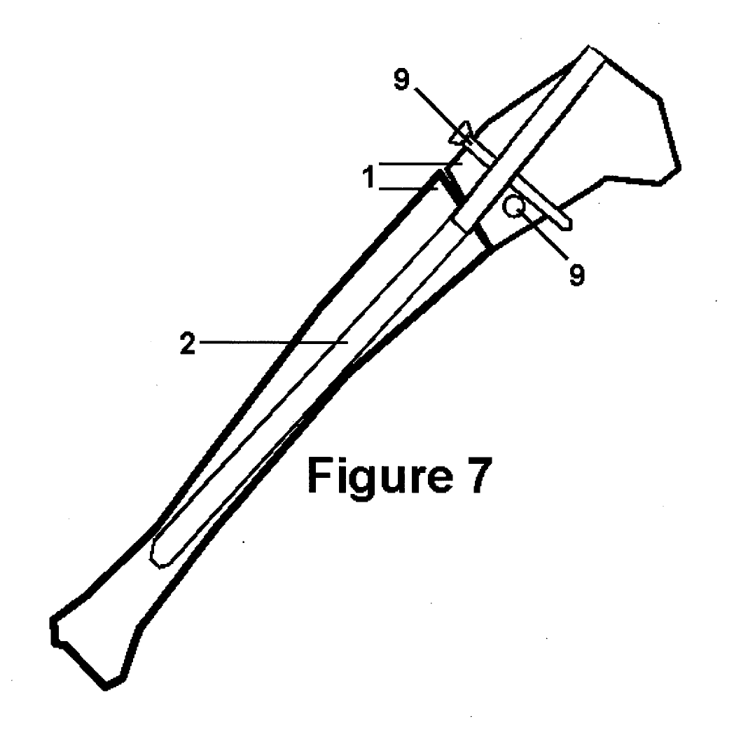

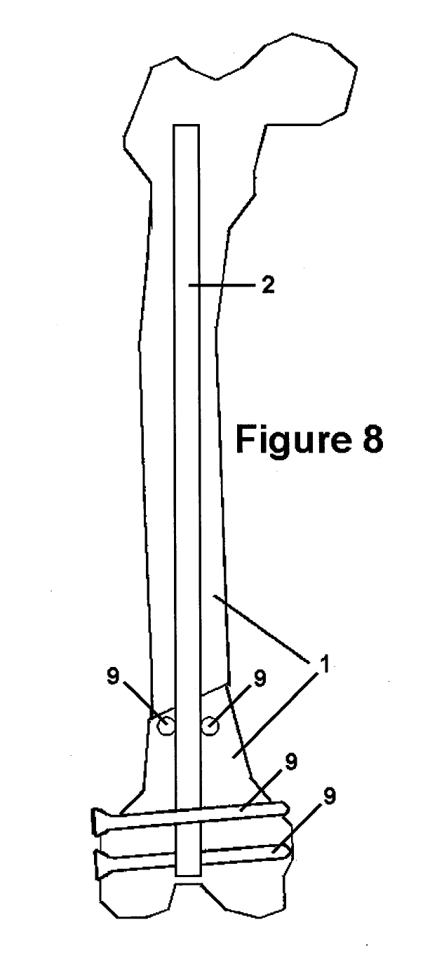

Once blocking screws 9 are properly inserted into the long bone 1, the blocking screw insertion device 10 may be removed. FIG. 7 shows a fractured long bone 1 which has been fixated by an intramedullary nail 2 guided by blocking screws 9, which were inserted using the blocking screw insertion device 10 of the present invention. Once the intramedullary nail has been locked into the fractured long bone 1 (using locking screws on each end of the nail) the blocking screws 9 may be removed. FIG. 8 shows an intramedullary nail 2 fixated femur fracture 1 in which the nail 2 has been guided by blocking screws 9. The blocking screw insertion device 10 of the present invention can be used with tibial nails, retrograde and anterograde femoral nails as well as humeral nails. For each specific nail type and manufacturer a specific blocking screw insertion device 10 can be created to provide easy insertion of blocking screws and accurate alignment of intramedullary nail fixation.

To use the blocking screw insertion device 10 of the present invention, one would select the proper blocking screw insertion device 10 for the specific long bone fracture to be repaired. That is, once the proper intramedullary nail to repair the specific long bone fracture is chosen, then the proper blocking screw insertion device 10 corresponding to the chosen intramedullary nail will be used to insert the blocking screws. The dummy nail head 2′ attached to the nail insertion handle 3, which is in turn connected to the blocking screw guide 6 directly or via the attachment means 5, is inserted into the medullary cavity. Once the dummy nail head 2′ is inserted into the medullary cavity, blocking screws 9 may be inserted into the proper locations in the long bone via a the alignment holes 8. Once the blocking screws are in place to blocking screw insertion device is removed. At this point, the intramedullary nail 2, attached to the nail insertion handle 3 can be inserted into the long bone fracture with substantially no malalignment of the nailed long bone. Once the intramedullary nail 2 is locked into the fractured long bone 1 using intramedullary nail locking screws (not shown), the blocking screws may be removed.

It is to be expected that considerable variations may be made in the embodiments disclosed herein without departing from the spirit and scope of this invention. Particularly, while the invention has been described with respect to the blocking screw guide 6 being in the form of a curved or arced plate, other forms, shapes and dimensions may be used with equal utility. Further, while the alignment means in the blocking screw guide 6 are depicted as alignment holes 8, other alignment means may be substitute for the holes as long as the function remains the same. Accordingly, the significant improvements offered by this invention are to be limited only by the scope of the following claims.

1-7. (canceled)

8. An intramedullary nailing method for the fixation of fractured long bones which substantially reduces or eliminates the incidence of malalignment of the of fractured long bones, said method comprising:
selecting the proper intramedullary nail for the specific long bone fracture;
providing a blocking screw insertion guide comprising:
an intramedullary nail dummy head consisting of a short segment of the proximal portion of said proper intramedullary nail;
a nail insertion handle specific to said proper intramedullary nail; and
a blocking screw guide portion;
said dummy head connected to said nail insertion handle in the same manner that said proper intramedullary nail would be connected for insertion into said fractured long bone;
said blocking screw guide portion attached to said nail insertion handle and including means to align/guide insertion of blocking screws into proper positions to substantially reduce or eliminate the incidence of malalignment of said fractured long bone during intramedullary nail fixation thereof;
inserting said dummy head of said blocking screw insertion guide into the medullary cavity of said fractured long bone; and
using said blocking screw guide portion to insert blocking screws into said fractured long bone.

9. The intramedullary nailing method of claim 8, further comprising the step of removing said dummy head of said blocking screw insertion guide from said medullary cavity of said fractured long bone after said step of inserting said blocking screws.

10. The intramedullary nailing method of claim 9, further comprising the step of inserting said proper intramedullary nail into said fractured long bone, said insertion of said proper intramedullary nail being guided by said blocking screws such that the incidence of malalignment of said fractured long bone is substantially reduced or eliminated after said step of removing said dummy head of said blocking screw insertion guide from said medullary cavity of said fractured long bone.

11. The intramedullary nailing method of claim 10, further comprising the step of locking said proper intramedullary nail into the medullary cavity of said fractured long bone using conventional locking screws or pins after said step of inserting said proper intramedullary nail into said fractured long bone.

12. The intramedullary nailing method of claim 11, further comprising the step of removing said blocking screws after said step of locking said proper intramedullary nail into the medullary cavity of said fractured long bone.
